SEQUENCE LISTING (7 sequences)

SEQ ID NO: 1 - DNA, 2685 bp, Clostridium difficile
atggacaacaaaacctactattacgatgaagatagcaaactggtaaaaggcctgatcaacatcaacaatagcctgttttacttcgacccgatcgagtttaacctggttactggttggcaaaccatcaacggtaagaagtattattttgatatcaatacgggtgcagccctgacgtcctacaaaatcattaacggcaaacatttctatttcaataacgacggtgttatgcagctgggcgtattcaaaggcccagatggttttgaatattttgcgccggcgaacacccagaacaacaacattgaaggtcaagctatcgtttaccagagcaaattcctgacgctgaacggtaaaaagtactatttcgacaacaactctaaagcggttaccggctggcgcatcattaacaacgagaaatactacttcaacccgaacaatgctatcgcagccgtgggtctgcaggtgattgataacaacaagtactactttaacccggacaccgctattatttctaaaggttggcagaccgttaatggtagccgttattacttcgataccgacaccgctatcgctttcaacggttataaaaccatcgacggcaagcacttttatttcgattctgattgcgttgttaaaatcggcgtgttctccacttctaacggttttgaatacttcgcaccggcaaacacctacaataacaatatcgaaggccaggcgattgtctaccagtccaaatttctgaccctgaatggcaaaaaatattacttcgacaacaattccaaagccgtcaccggttggcagactatcgactctaagaaatattattttaacaccaacactgcggaagcagcaactggttggcagacgattgacggcaagaagtactattttaacactaacactgcggaggcagcgaccggctggcagaccattgatggtaaaaagtattatttcaacactaacaccgcgattgcatctaccggttacaccatcatcaacggcaaacacttctacttcaacactgacggtatcatgcaaattggcgttttcaaaggtccgaacggtttcgaatacttcgccccagccaacacggacgcgaacaacatcgaaggtcaggcgattctgtaccagaatgagttcctgaccctgaacggcaagaaatactatttcggttccgattccaaagctgtaaccggctggcgtatcatcaacaacaaaaagtactacttcaatcctaacaacgcaatcgctgcgattcacctgtgtactatcaacaacgacaaatactatttttcttacgacggtatcctgcagaacggctatatcactatcgaacgtaacaacttctatttcgatgctaacaacgaaagcaaaatggtgacgggcgtgttcaaaggcccgaacggcttcgaatagtttgcacctgcaaacacccacaacaacaacattgagggtcaggcgatcgtttaccagaataaattcctgaccctgaacggtaagaaatattacttcgataacgacagcaaagccgttaccggctggcaaaccattgatggtaagaaatactactttaatctgaacaccgccgaagccgctactggttggcagacgatcgatggcaaaaagtactatttcaacctgaatactgcggaggcggcgaccggttggcagaccattgacggcaaaaagtattacttcaacacgaacacgttcatcgcatccactggttacaccagcattaacggtaaacacttctactttaacacggatggcattatgcaaattggtgtgtttaaaggtccaaacggttttgaatatttcgcaccggctaacacggacgctaacaatatcgaaggccaggctattctgtaccaaaacaaattcctgactctgaacggcaagaaatattattttggctctgattctaaagcggttacgggcctgcgtaccatcgacggtaaaaaatactacttcaacaccaacaccgctgtcgcagtaactggctggcagaccatcaacggtaaaaagtactacttcaataccaacaccagcatcgcttctacgggttatactatcatcagcggcaaacacttctatttcaacaccgacggtatcatgcagatcggcgttttcaaaggtccggacggtttcgaatacttcgctccggcgaataccgacgccaacaacatcgagggccaggctatccgttaccagaaccgttttctgtatctgcacgacaatatctattacttcggtaacaactctaaagctgcgaccggctgggttacgattgacggtaaccgctactacttcgaaccgaacaccgcgatgggtgcgaacggttacaaaaccatcgataacaaaaacttctatttccgtaacggcctgccacagatcggtgttttcaagggttctaatggttttgagtattttgcgccggcgaacactgacgctaacaacatcgaaggtcaggcgattcgttatcagaaccgtttcctgcatctgctgggcaagatttattacttcggcaacaacaccaaagcggtgactggctggcaaactattaatggtaaagtttactactttatgccagacactgctatggctgcagctggtggcctgttcgaaatcgacggcgttatttacttctttggcgttgacggcgtgaaagcgccgggtatttatggt

SEQ ID NO: 2 - protein, 895 aa, Clostridium difficile
MDNKTYYYDEDSKLVKGLININNSLFYFDPIEFNLVTGWQTINGKKYYFDINTGAALTSYKIINGKHFYFNNDGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 3 - DNA, 1611 bp, Clostridium difficile
atgatggtttctggcctgatctatattaacgattctctgtactatttcaaaccgccggttaacaacctgatcaccggtttcgtaactgttggcgacgataagtactactttaatccaattaacggtggtgcggccagcattggcgaaactatcatcgacgacaaaaactactacttcaatcagtccggtgttctgcagaccggtgtattctctaccgaagatggcttcaagtattttgcgccggctaacaccctggatgaaaacctggaaggtgaagctattgactttaccggcaaactgatcattgacgaaaatatctactacttcgacgataactaccgtggtgcagtagaatggaaagaactggacggtgaaatgcactacttctctccggaaactggtaaagcttttaagggcctgaaccagatcggtgattacaaatactacttcaacagcgatggtgtgatgcaaaaaggtttcgtgagcatcaatgataacaagcactactttgacgattccggcgtgatgaaagtcggctatactgaaatcgacggtaaacacttctactttgctgaaaacggcgaaatgcaaatcggcgttttcaatactgaagacggcttcaaatatttcgctcatcacaacgaagatctgggtaacgaagaaggcgaggagatttcctactccggcattctgaacttcaacaacaaaatttactactttgatgattctttcaccgccgtggttggctggaaagacctggaagatggttccaaatattatttcgatgaggataccgctgaagcgtacattggtctgtctctgatcaacgatggccagtactattttaacgacgacggcattatgcaggtgggcttcgttaccattaacgacaaagttttctattttagcgattccggtatcatcgaaagcggcgtgcagaacatcgatgataactacttctacatcgatgacaatggtatcgttcagatcggcgtattcgatacctctgacggttataaatacttcgccccggcgaacaccgttaacgacaacatttatggtcaggcagtcgagtactccggcctggttcgtgtgggtgaagacgtatactacttcggtgaaacgtacaccatcgagacgggctggatctacgacatggaaaacgaatctgacaaatattacttcaacccggaaactaaaaaggcgtgcaaaggcatcaacctgatcgatgacatcaaatactatttcgacgaaaaaggcatcatgcgcactggtctgatcagcttcgaaaacaacaactattatttcaacgagaatggcgagatgcagttcggttacatcaacatcgaagacaagatgttctactttggtgaagacggtgtaatgcagattggtgttttcaacacccctgacggttttaaatattttgcacatcagaacactctggacgagaacttcgagggtgagtctatcaactataccggctggctggacctggatgagaaacgttactacttcacggacgaatacattgcggcaaccggtagcgtgatcattgatggtgaagaatactattttgatccggacacggcacaactggtcatctctgaa

SEQ ID NO: 4 - protein, 537 aa, Clostridium difficile
MMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 5 - protein, 924 aa, Artificial Sequence
  AMINO ACIDS SEQUENCE OF LIPIDATED TOXIN A-RBD (rLIPO-A-RBD)
CSQEAKQEVKEAVQAVESDVKDTAGSHMDNKTYYYDEDSKLVKGLININNSLFYFDPIEFNLVTGWQTINGKKYYFDINTGAALTSYKIINGKHFYFNNDGVMQLGVFKGPDGFEYFAPANTQNNNIEGQAIVYQSKFLTLNGKKYYFDNNSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYGLE

SEQ ID NO: 6 - protein, 566 aa, Artificial Sequence
  AMINO ACIDS SEQUENCE OF LIPIDATED TOXIN B-RBD (rLIPO-B-RBD)
CSQEAKQEVKEAVQAVESDVKDTAGSHMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEESEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISELE

SEQ ID NO: 7 - protein, 27 aa, Artificial Sequence
  a lipid-box signal sequence
CSQEAKQEVKEAVQAVESDVKDTAGSH

(19) United States
(12) Patent Application Publication
(10) Pub. No.: US 2020/0123210 A1
(43) Pub. Date: Apr. 23, 2020

(54) IMMUNOGENIC PREPARATIONS AND METHODS AGAINST CLOSTRIDIUM DIFFICILE INFECTION

(71) Applicant: Eternity Biomedical Laboratories, Inc., Chupei (TW)

(72) Inventors: Chi-Chang Chen, Hsinchu County (TW); Pele Choi-Sing Chong, Hsinchu County (TW)

(73) Assignee: Eternity Biomedical Laboratories, Inc., Chupei, Hsinchu County (TW)

(21) Appl. No.: 16/655,765

(22) Filed: Oct. 17, 2019

Related U.S. Application Data
(60) Provisional application No. 62/746,769, filed on Oct. 17, 2018.

(51) Int. Cl. C07K 14/33; A61K 39/08; C07K 17/00; A61K 39/39; A61K 47/54; A61P 31/04

(52) U.S. Cl. CPC C07K 14/33; A61K 39/08; C07K 17/00; A61K 2039/505; A61K 47/543; A61P 31/04; A61K 39/39

(57) ABSTRACT

The present invention relates in general to the field of immunization, and particularly, an immunogenic preparation against Clostridium difficile infection (CDI), and a method for generating immunity against CDI by administering the immunogenic preparation to a subject in need. The present invention is useful for prevention and treatment of CDI and associated disease or disorder.

### RELATED APPLICATIONS

This application claims the benefit of U.S. provisional application No. 62/746,769, filed Oct. 17, 2018 under 35 U.S.C. § 119, the entire content of which is incorporated herein by reference.

### TECHNOLOGY FIELD

The present invention relates in general to the field of immunization, and particularly, an immunogenic preparation against Clostridium difficile infection (CDI), and a method for generating immunity against CDI by administering the immunogenic preparation to a subject in need. The present invention is useful for prevention and treatment of CDI and associated diseases or disorders.

### BACKGROUND OF THE INVENTION

Clostridium difficile infection (CDI) is a gram-positive spore-forming anaerobic bacterium which causes diseases in humans and animals such as pigs, horses and cattle. Specifically, due to the imbalance of intestinal micro-flora by antibiotic therapies used during hospitalization, CDI often causes the opportunistically nosocomial infection in hospitalized patients. CDI often results in Clostridium difficile-associated disease (CDAD), such as diarrhea, pseudomembranous colitis, and toxic megacolon [1,2]. As the significant increase in multi-drugs resistance, CDI has recently become a serious emerging infectious disease worldwide [3], which causes not only a risk to public health but also significant economic loss in livestock production.

The pathogenicity of C. difficile is largely mediated by two clostridial toxins, toxin A and toxin B (TcdA and TcdB), which are secreted in the gastrointestinal environment of infected hosts and disrupt the epithelial cell barriers in the small intestine [7]. Both toxins consist of holotoxins with multi-functional domains that mediate C. difficile pathogenesis. The mechanism underlying TcdA and TcdB toxicity involves three steps: (a) binding to unidentified receptor protein(s) on the surface of intestinal epithelium and internalization through its C-terminal receptor binding domain, (b) auto-cleavage and translocation of the N-terminal glucosyltransferase domain to the cytosol from the endosomal membrane; and (c) the N-terminal enzymatic region that inactivates the Rho GTPase family by glycosylation [7],[8].

Passive immunization with anti-toxin antibodies has been shown to confer protection against CDI in animal models and TcdA-specific monoclonal antibodies have been tested in clinical trials [13]-[15]. In addition, different C. difficile (Cd) vaccine strategies are evaluated; including vaccination with formalin-inactivated bacteria or Cd toxin A [16]-[19]. However, by means of formalin inactivation, the important protective epitopes of the antigens could be destroyed, leading to lack of protection in vivo as a result. Immunization with the receptor binding domain (RBD) of individual C. difficile toxin as an antigen formulated with different adjuvants has been shown to elicit toxin-neutralizing antibody responses and protect mice from toxin or Cd bacteria challenges [20]-[26]. In another study [31], a TcdB RBD was designed and expressed in E. coli. Recombinant TcdB RBD (B-rRBD) was purified, and found that it failed to induce sufficient protection against a lethal dose of C. difficile spores in the hamsters challenge model, especially in the absence of adjuvant.

There is still a need to develop an effective approach against CDI and associated diseases or disorders.

### SUMMARY OF THE INVENTION

In this invention, it is disclosed for the first time that a combination of receptor-binding domains (RBD) of toxin A (TcdA) and B (TcdB) of C. difficile, each of which is lipidated, is effective in producing protective immunity against Clostridium difficile infection (CDI).

Therefore, in one aspect, the present invention provides an immunogenic preparation against Clostridium difficile infection (CDI), comprising (i) a lipidated receptor-binding domain of C. difficile toxin A (lipo-A-RBD) polypeptide and (ii) a lipidated receptor-binding domain of C. difficile toxin B (lipo-B-RBD) polypeptide, in an amount effective to induce protective immunity against CDI.

In another aspect, the present invention provides a method for generating protective immunity against CDI in a subject in need, comprising administering to the subject an effective amount of an immunogenic preparation as described herein. The method of the present invention is also effective in treating or preventing a disease or disorder associated with CDI. Also provided is use of a preparation comprising (i) a lipidated receptor-binding domain of C. difficile toxin A (lipo-A-RBD) polypeptide and (ii) a lipidated receptor-binding domain of C. difficile toxin B (lipo-B-RBD) polypeptide for manufacturing a medicament (e.g. a vaccine) for generating protective immunity against CDI and for preventing a disease or disorder associated with CDI.

Specifically, the lipo-A-RBD polypeptide comprises a receptor-binding domain of C. difficile toxin A (A-RBD) polypeptide modified with a first lipid moiety, and/or the lipo-B-RBD polypeptide comprises a receptor-binding domain of C. difficile toxin B (B-RBD) polypeptide modified with a second lipid moiety.

In some embodiments, the first lipid moiety and the second lipid moiety are different or the same.

In some embodiments, each of lipid moieties comprises one or more lipid molecules selected from the group consisting of palmitoyl, stearoyl, decanoyl, and any combination thereof.

In some embodiments, the A-RBD polypeptide comprises an amino acid sequence at least 85% (e.g., 90%, 95%, 96%, 97%, 98% or 99%) identical to SEQ ID No: 2. In one embodiment, the A-RBD polypeptide comprises the amino acid sequence of SEQ ID NO: 2.

In some embodiments, the B-RBD polypeptide comprises an amino acid sequence at least 85% (e.g., 90%, 95%, 96%, 97%, 98% or 99%) identical to SEQ ID No: 4. In one embodiment, the B-RBD polypeptide comprises the amino acid sequence of SEQ ID NO: 4.

In some embodiments, the lipo-A-RBD polypeptide or the lipo-B-RBD polypeptide contains a lipid-box signal sequence at the N-terminal.

In some embodiments, the immunogenic preparation of the present invention further comprises a pharmaceutically acceptable carrier.

In some embodiments, the immunogenic preparation of the present invention includes a further component as an adjuvant.

In some embodiments, the immunogenic preparation of the present invention does not include a further component as an adjuvant.

Examples of a disease or disorder associated with CDI include but are not limited to diarrhea, pseudomembranous colitis, and toxic megacolon.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows the constructions of two plasmids expressing tcdA-RBD and tcdB-RBD in E. coli system. The nucleotide fragment sequences encoding rlipo-A-RBD (SEQ ID NO: 1) and rlipo-B-RBD (SEQ ID NO: 3) are listed in the text.

FIG. 2 shows the ELISA results (IgG titer against A-rRBD) that were determined with antisera obtained from different groups of mice immunized with different amounts of rlipo-A-RBD (A1)+rlipo-B-RBD (B1) combinations.

FIG. 3 shows the ELISA results (IgG titer against B-rRBD) that were determined with antisera obtained from different groups of mice immunized with different amounts of rlipo-A-RBD (A1)+rlipo-B-RBD (B1) combinations.

FIG. 4 shows the ELISA results (IgG titer against A-rRBD) that were determined with antisera obtained from different groups of hamsters immunized with different amounts of rlipo-A-RBD (A1)+rlipo-B-RBD (B1) combinations.

FIG. 5 shows the ELISA results (IgG titer against B-rRBD) that were determined with antisera obtained from different groups of hamsters immunized with different amounts of rlipo-A-RBD (A1)+rlipo-B-RBD (B1) combinations.

FIG. 6 shows C. difficile spore challenge in hamster model studies. Different groups of hamsters (n=6) were gastrically inoculated with >100 CFU of VPI10463 strain of C. difficile (the dose can kill >50% of hamsters) at 2 weeks after the third immunization with rlipo-A-RBD (A1)+rlipo-B-RBD (B1) combinations. The final survival rates were reported.

FIG. 7 shows C. difficile spore challenge in hamster model studies. Different groups of hamsters (n=6) were gastrically inoculated with >100 CFU of different strains (VPI10463, 630, R20291 and M120) of C. difficile (the dose can kill >50% of hamsters) at 2 weeks after the third immunization with rlipo-A-RBD (A1)+rlipo-B-RBD (B1) combinations. The final survival rates were reported.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “polypeptide” refers to a polymer composed of amino acid residues linked via peptide bonds. For example, a polypeptide can be a polymer composed of linked amino acids e.g. about 1,000 amino acids or less in length.

As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of ±10% around the cited value.

As used herein, the term “pharmaceutical preparation” can refer to pharmaceuticals in any forms, for example, a composition, a combination or a kit. A composition can refer to a homogenous mixture, for example, in a form e.g. tablets, capsules, pills, powders, granules, solutions, suspensions and emulsions and any pharmaceutical acceptable forms. A combination can refer to a product obtained from combining two or more active ingredients which are present physically separately in one or more packaging units for time-sequential administration. A kit can refer to a collection or set of the aforementioned pharmaceutical preparation, preferably, provided in separate form within a single container. The container, also preferably, comprises instructions for using such pharmaceutical preparation or carrying out the methods of the present invention.

As used herein, the term “individual” or “subject” includes human or non-human animals, for example, companion animals (such as dogs, cats and the like), farm animals (such as cattle, sheep, pigs, horses, etc.), or laboratory animals (such as rats, mice, guinea pigs, etc.).

As used herein, “corresponding to,” refers to a residue at the enumerated position in a protein or peptide, or a residue that is analogous, homologous, or equivalent to an enumerated residue in a protein or peptide.

As used herein, the term “substantially identical” refers to two sequences having more than 85%, preferably 90%, more preferably 95%, and most preferably 100% homology.

As used herein, the term “lipopeptide” or “lipidated polypeptide” as used herein refers to a polypeptide, preferably an immunogenic polypeptide, modified with (e.g. covalently linked to) a lipid residue or moiety.

As used herein, the term “Toxin A (TcdA)” refers to a toxin A polypeptide from C. difficile. The term “TcdA-RBD” or “A-RBD” or “A-RBD polypeptide” refers to the receptor binding domain of Toxin A. A A-RBD polypeptide described herein can be a naturally occurring protein of any suitable species e.g. VPI 10463, 630 and R20291. In some embodiments, a A-RBD polypeptide as described herein includes an amino acid sequence set forth in SEQ ID NO: 2 (VPI 10463). In some other embodiments, a A-RBD polypeptide as described herein may be a naturally occurring protein that is highly homologous to SEQ ID NO:2, for example, sharing at least 85% sequence identity in the entire length (e.g., at least 90%, at least 93%, at least 95%, or at least 97%). Such A-RBD polypeptide can be readily identified from publically available gene database (e.g., GenBank) using SEQ ID NO:2 as a query.

As used herein, the term “Toxin B (TcdB)” refers to a toxin B polypeptide from C. difficile. The term “TcdB-RBD” or “B-RBD” or “B-RBD polypeptide” refers to the receptor binding domain of Toxin B. A B-RBD polypeptide described herein can be a naturally occurring protein of any suitable species e.g. VPI 10463, 630 and R20291. In some embodiments, a B-RBD polypeptide as described herein includes an amino acid sequence set forth in SEQ ID NO: 4 (VPI 10463). In some other embodiments, a B-RBD polypeptide as described herein may be a naturally occurring protein that is highly homologous to SEQ ID NO:4, for example, sharing at least 85% sequence identity in the entire length (e.g., at least 90%, at least 93%, at least 95%, or at least 97%). Such B-RBD polypeptide can be readily identified from publically available gene database (e.g., GenBank) using SEQ ID NO: 4 as a query.

To determine the percent identity of two amino acid sequences, the sequences are aligned for optimal comparison purposes (e.g., gaps can be introduced in the sequence of a first amino acid sequence for optimal alignment with a second amino acid sequence). In calculating percent identity, typically exact matches are counted. The determination of percent homology or identity between two sequences can be accomplished using a mathematical algorithm known in the art, such as BLAST and Gapped BLAST programs, the NBLAST and XBLAST programs, or the ALIGN program.

It is understandable that a polypeptide may have a limited number of changes or modifications that may be made within a certain portion of the polypeptide irrelevant to its activity or function and still result in a variant with an acceptable level of equivalent or similar biological activity or function. The term “acceptable level” can mean at least 20%, 50%, 60%, 70%, 80%, or 90% of the level of the referenced protein as tested in a standard assay as known in the art. Biologically functional variant polypeptides are thus defined herein as those polypeptides in which certain amino acid residues may be substituted. Polypeptides with different substitutions may be made and used in accordance with the invention. Modifications and changes may be made in the structure of such polypeptides and still obtain a molecule having similar or desirable characteristics. For example, certain amino acids may be substituted for other amino acids in the peptide/polypeptide structure without appreciable loss of activity.

The polypeptide of the present invention may be produced by chemical synthesis using techniques well known in the chemistry of proteins such as solid phase synthesis or synthesis in homogenous solution.

Typically, the polypeptide of the present invention may be prepared using recombinant techniques. In this regard, a recombinant nucleic acid comprising a nucleotide sequence encoding a polypeptide of the present invention and host cells comprising such recombinant nucleic acid are provided. The host cells may be cultured under suitable conditions for expression of the polypeptide of interest. Expression of the polypeptides may be constitutive such that they are continually produced or inducible, requiring a stimulus to initiate expression. In the case of inducible expression, protein production can be initiated when desired by, for example, addition of an inducer substance to the culture medium, for example, isopropyl β-D-1-thiogalactopyranoside (IPTG) or methanol. Polypeptide can be recovered and purified from host cells by a number of techniques known in the art, for example, chromatography e.g., HPLC or affinity columns.

In some embodiments, the polypeptide of the present invention can be said to be “isolated” or “purified” if it is substantially free of cellular material or chemical precursors or other chemicals that may be involved in the process of peptide preparation. It is understood that the term “isolated” or “purified” does not necessarily reflect the extent to which the polypeptide has been “absolutely” isolated or purified e.g. by removing all other substance s (e.g., impurities or cellular components). In some cases, for example, an isolated or purified polypeptide includes a preparation containing the peptide having less than 50%, 40%, 30%, 20% or 10% (by weight) of other proteins (e.g. cellular proteins), having less than 50%, 40%, 30%, 20% or 10% (by volume) of culture medium, or having less than 50%, 40%, 30%, 20% or 10% (by weight) of chemical precursors or other chemicals involved in synthesis procedures.

As used herein, the term “a lipo-A-RBD polypeptide” refers to an A-RBD polypeptide modified with a lipid moiety (containing at least one preferably two or more lipid molecules).

As used herein, the term “a lipo-B-RBD polypeptide” refers to a B-RBD polypeptide modified with a lipid moiety (containing at least one preferably two or more lipid molecules).

In some embodiments, a lipo-A-RBD polypeptide or a lipo-B-RBD polypeptide as described herein contains a lipid-box signal sequence at the N-terminal. This signal peptide can be remodified during the lipidation process by E. coli enzymes. Bacterial lipoproteins (BLPs) are characterized by the presence of a lipobox motif, which is located in the C-terminal part of their leader peptide and contains a conserved cysteine residue, which is the target for N-acyl-S-diacylglyceryl-cysteinyl modification (Hantke & Braun, 1973). Modification of the precursor protein is mediated by the consecutive activity of three enzymes: the phosphatidylglycerol-preprolipoprotein diacylglyceryl transferase responsible for adding a diacylglycerol residue to the thiol group of the lipobox cysteine, the prolipoprotein signal peptidase/signal peptidase II which subsequently cleaves the lipidation signal sequence and the phospholipid-apolipoprotein N-acyltransferase which completes lipid modification (Hantke & Braun, 1973; Rezwan, Grau, Tschumi, & Sander, 2007).

According to the present invention, an effective amount of the active ingredient may be formulated with a physiologically (or pharmaceutically) acceptable carrier into a composition of an appropriate form for the purpose of delivery or storage. The composition of the present invention particularly comprises about 0.1% by weight to about 100% by weight of the active ingredient, wherein the percentage by weight is calculated based on the weight of the whole composition. In some embodiments, the composition of the present invention can be a pharmaceutical composition or medicament for treatment or an immunogenic composition for generating anti-viral immunity.

As used herein, the term “acceptable” can mean that the carrier is compatible with the active ingredient in the composition, and preferably can stabilize said active ingredient and is safe to the receiving individual. Said carrier may be a diluent, vehicle, excipient, or matrix to the active ingredient. Acceptable carriers may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, phenol, butyl or benzyl alcohol; catechol; resorcinol; cyclohexanol; 3-pentanol; and m-cresol); low molecular weight (less than about 10 residues) polypeptides; proteins, such as serum albumin or gelatin; hydrophilic polymers such as polyvinylpyrrolidone; amino acids such as glycine, glutamine, asparagine, histidine, arginine, or lysine; chelating agents such as EDTA; sugars such as glucose, sucrose, mannitol, trehalose or sorbitol; and/or surfactants such as polyoxyethylenesorbitans (e.g., TweenTM 20, 40, 60, 80 or 85), and other sorbitans (e.g. Span.TM. 20, 40, 60, 80 or 85) or polyethylene glycol (PEG). The composition of the present invention can provide the effect of rapid, continued, or delayed release of the active ingredient after administration to the patient.

The compositions to be used as a pharmaceutical composition for in vivo administration are typically sterile. This may be accomplished by, for example, by filtration through sterile filtration membranes. In some embodiments, therapeutic compositions may be placed into a container having a sterile access port, for example, an intravenous solution bag or vial having a stopper pierceable by a hypodermic injection needle.

In some embodiments, the immunogenic preparation of the present invention comprising a combination of a lipo-A-RBD polypeptide and a lipo-B-RBD polypeptide can further comprise an adjuvant. Typical examples of adjuvants to enhance effectiveness of a vaccine composition include, but are not limited to, aluminum salts, oil-in-water emulsion formulations, saponin adjuvants, complete Freund's adjuvant (CFA) and incomplete Freund's adjuvant (IFA).

In some embodiments, the preparation of the present invention does not include a further component as an adjuvant.

According to the present invention, a lipo-A-RBD polypeptide and a lipo-B-RBD polypeptide are present at a proper ratio of 0.1:1 to 1:0.1 (by weight) in the immunogenic preparation of the present invention. In some embodiments, the ratio is about 1:1 (by weight).

The preparation described herein may be in unit dosage forms such as tablets, pills, capsules, powders, granules, solutions or suspensions, or suppositories. The preparation can be administered to a subject (e.g., a human) in need of the treatment via a suitable route such as orally, parenterally (e.g. intramuscularly, intravenously, subcutaneously, and intraperitoneally), nasally, rectally, transdermally or inhalationally.

In particular, injectable preparation may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). Physiologically acceptable excipients may include, for example, 5% dextrose, 0.9% saline, Ringer's solution or other suitable excipients. In some embodiments, intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, the immunogenic preparation of the present invention can be provided as a particulate system. The particulate system can be a microparticle, a microcapsule, a microsphere, a nanocapsule, or similar particle.

The present invention provides a method for generating immunity against CDI by administering to a subject in need an effective amount of a preparation comprising a lipo-A-RBD polypeptide and a lipo-B-RBD polypeptide as described herein. The method of the present invention is useful in prevention and treatment of CDI and associated diseases.

Examples of diseases or conditions associated with CDI include, without limitation, diarrhea, pseudomembranous colitis, and toxic megacolon.

To practice the method disclosed herein, an effective amount of an immunogenic preparation described herein can be administered to a subject (e.g., a human) in need of the treatment via a suitable route. “An effective amount” as used herein refers to the amount of each active agent required to confer therapeutic effect on the subject, either alone or in combination with one or more other active agents. In some embodiments, the “effective amount” used herein can be the amount of the lipopeptides sufficient to generate or induce an immune response against a pathogen (e.g. C. difficile) or an antigen (e.g., toxins A or B of C. difficile) in the recipient thereof. The lipopolypeptides as described herein can be administered to a subject in need simultaneously or sequentially. The term “immune response” may include, but is not limited to, a humoral response and a cell mediated immune response e.g. CD⁴⁺ or CD⁸⁺ cell activation. In some embodiments, the “effective amount” used herein can be the amount of an antibody to, for example, sufficient to target to the corresponding pathogen (e.g. C. difficile) and to treat associated diseases or conditions. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and may be addressed with no more than routine experimentation. In some embodiments, a maximum dose of the individual components or combinations thereof may be used, that is, the highest safe dose according to sound medical judgment. It will be understood by those of ordinary skill in the art that a patient may insist upon a lower dose or tolerable dose for medical reasons, psychological reasons or for virtually any other reason.

The subject to be treated by the methods described herein can be a mammal, more preferably a human. A human subject who needs the treatment may be a human patient having, at risk for, or suspected of having a target disease/disorder, such as C. difficile infection. A subject suspected of having any of such target disease/disorder might show one or more symptoms of the disease/disorder. A subject at risk for the disease/disorder can be a subject having one or more of the risk factors for that disease/disorder. A subject susceptible to CDI can be identified by methods known in the art and administered a composition of the invention. The dose of the composition depends, for example, on the particular antigen, whether an adjuvant is co-administered, and the type of adjuvant co-administered, the mode and frequency of administration, as can be determined by one skilled in the art. Administration is repeated as necessary, as can be determined by one skilled in the art. For example, a priming dose can be followed by two or three booster doses at weekly intervals or every two weeks.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Examples

To develop an effective recombinant subunit vaccine against CDI, in this invention, A-rRBD and B-rRBD were lipidated (rlipo-A-RBD and rlipo-B-RBD) and expressed in E. coli. The purified rlipo-A-RBD and rlipo-B-RBD were further characterized immunologically and found to be highly efficacious vaccine candidates against CDAD and not require formulations with other adjuvants.

1. Material and Methods

1.1 Constructs for Producing rlipo-RBD

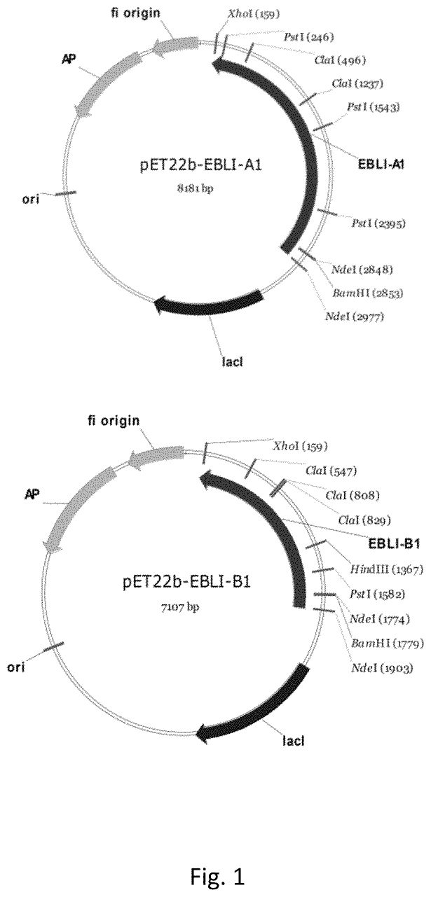

The plasmid containing rlipo-A-RBD was constructed based on the pET-22b (+) vector using NedI and Xho I sites as previously described [32]. Using a similar method, the plasmid containing rlipo-B-RBD was constructed as well. See FIG. 1. In brief, the 3′-end of either A-rRBD or B-rRBD was fused with the sequence containing a polyhistidine tag and XhoI restriction enzyme site [30]. The 5′ terminus was fused to an E. coli. lipid-box signal sequence by BamHI restriction enzyme site [32]. The 5′-end of lipid leader sequence also contained an NdeI restriction enzyme site. Finally, both A-rRBD and B-rRBD nucleotide sequences possessing 5′-lipid leader sequence and 3′ polyhistidine sequence containing NdeI and XhoI sites, respectively, were individually cloned into pET-22b(+) vector (Novagen, Darmstadt, Germany) by the NdeI and XhoI restriction enzyme sites. The pET-22b(+)_rlipo-A-RBD construct or pET-22b(+)_rlipo-B-RBD construct was transformed into E. coli C43 (DE3) (Imaxio; Saint-Beauzire, France) for either rlipo-A-RBD or rlipo-B-RBD expressions.

1.2 Production of rlipo-RBD

The pET-22b(+)_rlipo-A-RBD construct or pET-22b(+)_rlipo-B-RBD construct was transformed into E. coli C43 (DE3) and the transformed bacterial cells were cultured in LB Broth containing 100 μg/ml ampicillin (Imaxio; Saint-Beauzire, France). Once OD₆₀₀ₙₘ of bacteria culture achieved approximately 0.5, 1 mM isopropyl-β-D-thiogalacto-pyranoside (IPTG) was added into the culture medium to incubate at 20° C. for 16 hours. Bacteria were harvested by centrifugation and stored at −20° C. before lysis. Bacterial pellet was suspended in lysis buffer (50 mM Tris-Cl, pH8.0 containing 500 mM NaCl) and disrupted physically by French Press (Constant System, Daventry, UK) at 27 Kpsi. The cell lysate was pelleted and extracted twice with 50 mM Tris-Cl, pH8.0 containing 0.5% Triton X-100. The crude-extracted solution was purified by two step affinity chromatograph. First, nickel resin was used to separate any impurities. The eluent was dialyzed to remove imidazol and applied to an immobilized metal affinity chromatography (IMAC) (GE Healthcare, Uppsala, Sweden) charged with copper ion for LPS removal. All purification steps were performed at 4° C. and analyzed by 8% SDS-PAGE. Affinity chromatography was performed according to manufacturer's instruction. The residual endotoxin was determined by LAL assay (Associates of Cape Cod, Inc., Cape Cod, Mass.). The eluent was dialyzed in a 30 kDa cut-off dialysis bag against phosphate buffered saline (PBS), pH 7.2 containing 15% glycerol, and stored at −80° C. In all experiments, protein quantification was determined by BCA Protein Assay Kit (Thermo Pierce). Samples separated in the gel were transferred onto PVDF membrane (GE). PVDF membrane was blocked with 5% nonfat dry milk (w/v) in PBS for 1 hour. To specifically identify rlipo-B-RBD, the membrane was inoculated with anti-his tag (AbD Serotec; Kidlington, UK) or anti-TcdB antibodies [31,32] in PBS containing 1% nonfat dry milk (w/v) for 1 hour. To specifically identify rlipo-A-RBD, the membrane was inoculated with anti-his tag (AbD Serotec; Kidlington, UK) or anti-TcdA antibodies [31,32] in PBS containing 1% nonfat dry milk (w/v) for 1 hour. After washing twice with PBST (PBS containing 0.05% Tween 20), HRP-conjugated secondary antibodies in PBS containing 1% milk was added and incubated for 1 hour. Membrane was washed twice with PBST and developed using Luminata Crescendo substrate according to manufacturer's instruction (Millipore, Billerica, Mass.). The lipid moiety of rlipo-RBD was also analyzed using mass spectroscopy [32].

1.3 Immunogenicity Studies in Animals

Groups of mice (6 BALB/c mice per group) were vaccinated with three intramuscular injections of various amounts of either (a) rlipo-A-RBD (1, 3 or 10 μg) or (b) A-rRBD (3, 10 or 30 μg), (c) rlipo-B-RBD (1, 3 or 10 μg) or (d) B-rRBD (3, 10 or 30 μg) every two weeks. Before each immunization (week 0, week 2, week 4 and week 6), mice were bled by tail vein to collect sera that were stored at −20° C. before use in anti-RBD antibody titer determination using RBD-specific ELISA.

Groups of two NZW rabbits or six hamsters per group were intramuscularly vaccinated with 10 μg of either rlipo-A-RBD and/or rlipo-B-RBD formulated with and without alum three times, 14 days apart. Before each immunization (week 0, week 2, week 4 and week 6), animals were bled via the central ear artery. Sera were collected and stored at −20° C. for further analyses.

1.4 Antigen-Specific Enzyme-Linked Immunosorbent Assay (ELISA)

ELISA plate wells were coated either with 100 ng of A-rRBD, B-rRBD at 4° C. overnight, and then blocked with 5% nonfat dry milk (w/v) in PBS. Mouse antisera 2-fold serially diluted with PBS containing 1% BSA (Calbiochem, Darmstadt, Germany) were added to the wells followed by incubation at room temperature (RT) for 2 hours. After washing with 3×PBST, either anti-IgG isotypes (Invitrogen, Carlsbad, Calif.) or anti-IgA (Invitrogen, Carlsbad, Calif.) HRP-conjugated IgG (KPL, Gaithersburg, Md.) specific antibodies diluted in PBS containing 1% BSA were added to the wells and incubated at RT for 1 hour. After washing with 3×PBST, the plates were treated with TMB peroxidase substrate (KPL) at room temperature in the dark for 20 min. To determine anti-A-rRBD or anti-B-rRBD titer, OD₄₅₀ₙₘ absorbance was measured using a spectrophotometer (Spectra max M2, Molecular Devices, Sunnyvale, Calif.).

1.5 Neutralization Assay Against C. difficile Toxin (Tcd) A or B

The anti-TcdA or anti-TcdB neutralization assay was performed according to the protocol previously described by Huang et al. [31]. Briefly, Vero cells (2×10⁴ per well) were seeded into 96-well plates containing VP-SFM culture medium (Invitrogen, Carlsbad, Calif.) and 4 mM glutamine at 37° C., and allowed to grow to confluent. Mouse sera from mice immunized either with rlipo-A-RBD, or rlipo-B-RBD, A-rRBD or B-rRBD were serially diluted two-fold with fresh VP-SFM. 40 pg/mL of TcdA or TcdB (The Native Antigen Company Ltd, Oxfordshire, UK) was incubated at room temperature for 1 hour. The mixture containing mouse sera and TcdA or TcdB was added to the 96-well plates containing Vero cells and incubated at 37° C. for 24 hours. Anti-TcdA neutralization titers were calculated as the highest serum dilution which could protect 50% of cells from rounding due to toxin cytotoxicity. Cellular toxicity was recorded using a microscope equipped with a camera.

1.6 Preparation of Different Strain C. difficile Spores

The protocol for preparation of C. difficile spores was modified from the method previously reported by Lyras et al., [34]. Briefly, C. difficile strains VPI10463, CD196, 630, RD20291 and M120 were individually streaked on 10 anaerobic blood agar plates and grown anaerobically at 37° C. to induce sporulation at around the 5th or 6th day. The cells were harvested with disposable loops and washed in 10 mL PBS, and heat-shocked at 56° C. for 30 min to kill surviving vegetative cells. The spores were collected by low-speed centrifugation and resuspended in DMEM, aliquoted and frozen at −80° C. The frozen spores were then quantified before use by plating ten-fold serial dilutions of the spores onto Taurocholatefructose-agar (TFA) plates which were prepared with agar plus taurocholate-cefoxitin-cycloserinefructose-agar (TCCFA) without cycloserine and cefoxitin.

1.7 Challenges Studies in Animals

Hamster challenge model was performed as follows. Groups of hamsters were vaccinated with three intramuscular injections of various test immunogens, optionally formulated with either 300 μg of aluminum phosphate (alum) or Pam3CSK4 (InvivoGen, San Diego, Calif.), every two weeks. Before each immunization, hamster blood sera were carefully collected by the heart puncture and stored at −20° C. before use in anti-RBD antibody titer determination using RBD-specific ELISA. After three immunizations as described above, hamsters were given clindamycin orogastrically (30 mg/kg) to render them susceptible to C. difficile infection (day 0). On day-5 post clindamycin treatment, hamsters in each group were gastrically inoculated with 100 cell forming unit (CFU) of C. difficile spore, and monitored twice daily for 5 days and then daily thereafter. Animal bedding was changed and faecal pellets were collected every two days. Specimens were inoculated onto selective TCCFA plates and incubated anaerobically at 37° C. to determine if they were colonized with C. difficile. Faecal pellets were collected every two days for 12 days, then weekly until the study terminated (at least 14 days). Each hamster group was assessed for C. difficile colonization and survival rate.

To further evaluate the roles of anti-toxin neutralizing antibodies in vivo, different strains of C. difficile spore hamster challenge model was performed as described above. Two groups of hamsters (n=6) were vaccinated 3 times with PBS 2 weeks apart (one group is used for challenge as the positive control and one group has no challenge as the negative control) and another three separated groups of hamsters were immunized with either 3×3 μg of (rlipo-A-RBD (A1)+rlipo-B-RBD (B1)) or 3×10 μg of (rlipo-A-RBD (A1)+rlipo-B-RBD (B1)) intramuscularly. A week after the third immunization, blood samples collected from immunized hamster were assayed for anti-TcdA neutralizing antibody titers.

2. Results

2.1 Preparation of rlipo-RBD

Lipidated (rlipo-A-RBD (A1) or rlipo-B-RBD (B1)) were successfully expressed in E. coli C43 (DE3) strain and purified using Ni-affinity chromatography. The purified rlipo-A-RBD (A1) shows an expected molecular weight closed to 100 kDa (>85% purity), and rlipo-B-RBD (B1) shows an expected molecular weight closed to 75 kDa (>90% purity) as confirmed by SDS-PAGE. Most of the E. coli proteins and endotoxin (LPS) were successfully removed by the second IMAC-affinity column and washing with PBS containing 0.1% Triton-X100. The purity of eluted rlipo-RBD was confirmed by SDS-PAGE and the western blot analysis using a TcdA- or TcdB-specific monoclonal antibody. In any event, at least 5 to 10 mg of highly purified rlipo-RBD was easily obtained from 1 liter of bacteria culture.

2.2 Identification of Lipid Moiety of rlipo-RBD

The lipid moiety of rlipo-RBD was identified using mass spectroscopy analysis [32]. The purified rlipo-RBD was digested with trypsin and the tryptic fragments were analyzed using MALDI-TOF. Typical groups of ion mass peaks which exhibit the post-translational modification signature of recombinant lipoprotein, contain three peaks with m/z values of 1452, 1466, and 1480. The mass differences between these peaks are 14 amu and the pattern of isotopes in each group is exactly identical to that previous report [32]. The circular dichroism (CD) secondary structure analysis of both rlipo-A-RBD (A1) and rlipo-B-RBD (B1) were also performed and found that rlipo-A-RBD (A1) had correctly folded to form β-sheet structure similar to A-rRBD (>43%) [30]. This result is consistent with other reports that RBD forms stable folded β-solenoid secondary structures independently of other functional domains in the TcdA [30,31]. The best condition for preserving rlipo-A-RBD (A1) was to store the protein at 1 mg/mL in PBS containing 10% (v/v) of glycerol at −80° C. rlipo-B-RBD (B1) was found to be more stable than rlipo-A-RBD (A1) when it was stored at −80° C.

2.3 A Combination of rlipo-A-RBD and rlipo-B-RBD Elicited Strong Immunogenicity and Neutralizing Antibodies

2.3.1 Immunogenicity Studies

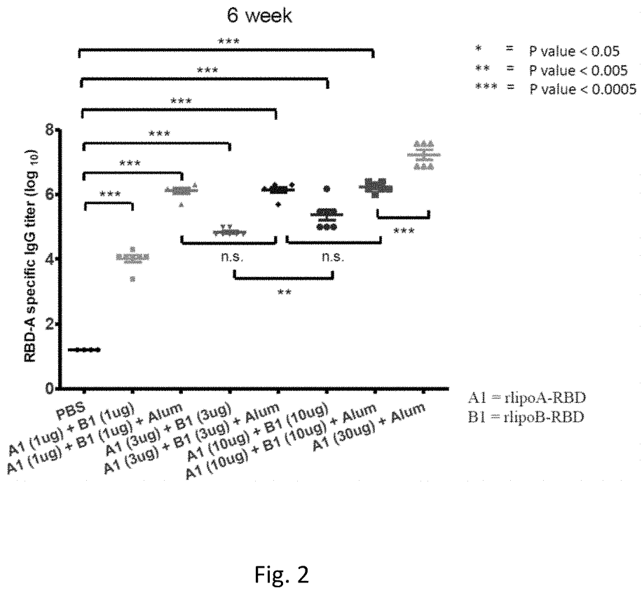

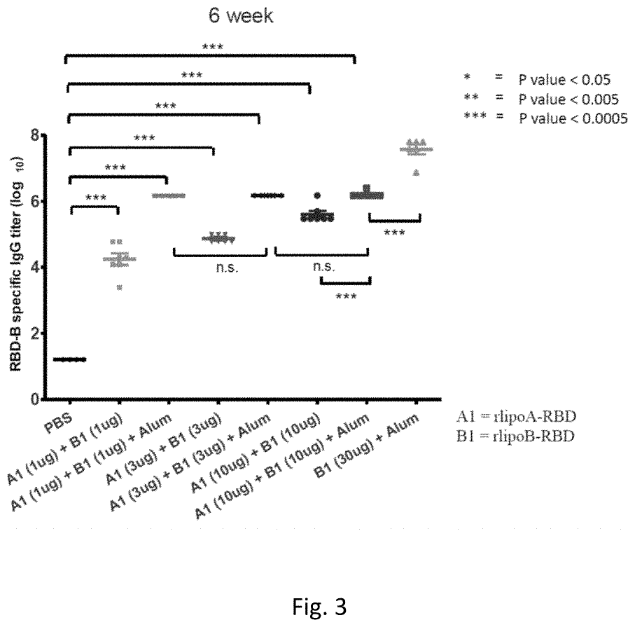

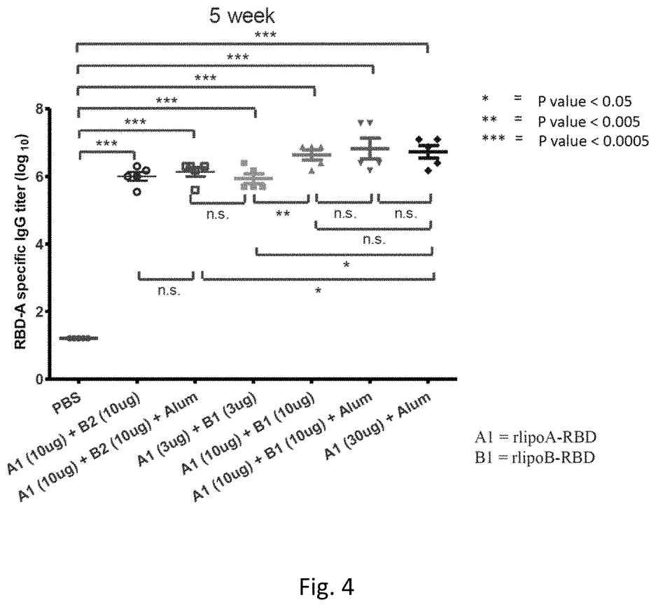

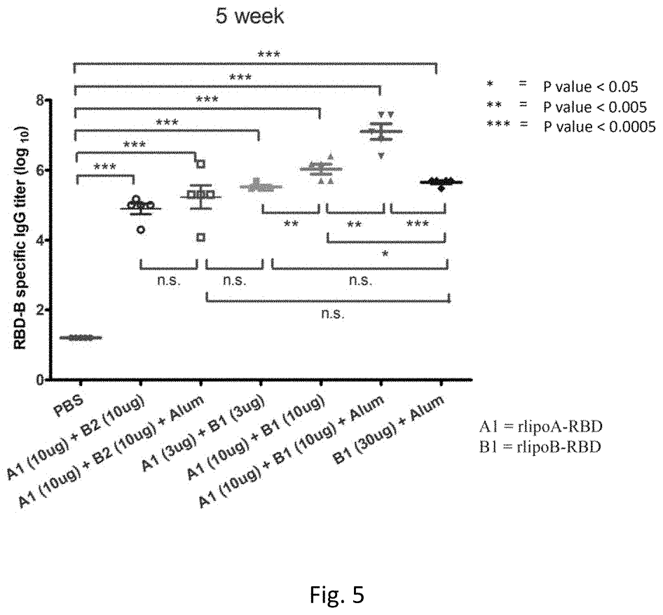

Mice were immunized with a combination of rlipo-A-RBD (A1) and rlipo-B-RBD (B1), and mouse antisera were collected and analyzed for the titers of antibodies against RBD-A or RBD-B by ELISA, respectively. Analyses of mouse antisera from each immunization using RBD-specific ELISA revealed that three doses of 1 μg of rlipo-A-RBD (A1) plus 1 μg of rlipo-B-RBD (B1), without alum, already induced very strong anti-RBD IgG antibody responses. In addition, antisera obtained from mice and hamster vaccinated with 3 doses of 3 μg of rlipo-A-RBD (A1) and 3 μg of rlipo-B-RBD (B1), and 3 doses of 10 μg of rlipo-A-RBD (A1) and 10 μg of rlipo-B-RBD (B1), without alum, was capable of inducing about 10⁵ or higher anti-RBD IgG titer; and when alum is added, the titer of the resultant antisera was further promoted to reach to 10⁶ or higher, which is deemed to be valuable in anti-toxin neutralization. Especially, a lower dose combination (3 μg of rlipo-A-RBD (A1) and 3 μg of rlipo-B-RBD (B1)), without alum, was capable to induce above 10⁵ anti-RBD IgG titer, which is comparable or better than a higher dose combination (10 μg of rlipo-A-RBD (A1) and 10 μg of B-rRBD (B2), with or without alum (FIGS. 4 and 5). These results strongly support a combination of rlipo-A-RBD (A1) and rlipo-B-RBD (B1) effective in inducing synergistic immunogenic effects against both RBD-A or RBD-B and potential as a good vaccine candidate. See FIGS. 2-5.

2.3.2 Anti-Toxin Neutralization Assay

To determine whether in various animals, a combination of rlipo-A-RBD (A1) and rlipo-B-RBD (B1) can generate antisera functionally neutralizing the cytotoxicity of C. difficile TcdA and TcdB, mouse, hamster and rabbit antisera were tested in a Vero cell cytotoxicity assay as described above. Table 1 shows the results.

In groups (1) and (2), antisera of animals immunized with 3×10 μg of B-rRBD (B2) exhibits a very low neutralizing antibody titer (1/16) and even if a extremely high dose of B-rRBD (B2) (3×30 μg) was used, the titer was still low (less than 1/32). The results suggest that B-rRBD (B2) alone is ineffective to induce functional antisera. In group (3), when B-rRBD (B2, 30 μg) is used with rlipo-A-RBD (A1, 10 μg), the antisera titer against toxin A achieved 1/512, but the antisera titer against toxin B was still relatively low (1/128 or less) even the dose of B-rRBD (B2, 30 μg) was 3-fold higher than that of rlipo-A-RBD (A1, 10 μg). Similar results were observed even when alum was added; in group (4), when the same combination, B-rRBD (B2, 30 μg) and rlipo-A-RBD (A1, 10 μg) was used in the presence of alum, the antisera titer against toxin A was further promoted to reach above 1/1024, but the antisera titer against toxin B was still relatively low (1/512 or less) even the dose of B-rRBD (B2, 30 μg) was 3-fold higher than that of rlipo-A-RBD (A1, 10 μg). The results suggest that B-rRBD (B2) can hardly induce functional antisera at a desired titer even when an extremely higher dose of B-rRBD (B2) was used and alum was further added.

In groups (5) and (6), it was unexpectedly found that rlipo-B-RBD (B1) at a relatively lower dose, 3×1 μg or 3×3 μg, was already capable of inducing functional antisera against toxin B, achieving the titer being 1/512 or 1/1024 or higher. Then, in groups (7)-(10), various combinations of rlipo-B-RBD (B1) plus rlipo-A-RBD (A1), B1 at different doses of 3 μg, 10 μg or 30 μg together with A1 at 10 μg, were tested in animals and confirmed that such combination were capable of inducing functional antisera against both toxin B and toxin A, achieving the titer being 1/512 or 1/1024 or higher. In particular, in group (7), the results show that 10 μg rlipo-A-RBD (A1) together with 3 μg of rlipo-B-RBD (B1) only induced antisera against toxin A at the titer 1/512, while when the same dose of rlipo-A-RBD (A1, 10 μg) was used together with a higher dose of rlipo-B-RBD (B1, 10 μg), the titer of the antisera against toxin A was promoted to reach to 1/1024 or higher. The results show that rlipo-B-RBD (B1) promotes the immunogenicity of rlipo-A-RBD (A1). Further, in groups (8) and (10), the same dose of rlipo-A-RBD (A1, 10 μg) and rlipo-B-RBD (B1, 10 μg), without or together with alum, show the antisera titer at a similar level. It indicates that a combination of rlipo-A-RBD (A1) and rlipo-B-RBD (B1) per se is sufficient to generate antisera functionally neutralizing toxin A and toxin B, even in the absence of alum.

| | | Anti-toxin Neutralizing Antibody Responses (Titers) | | | | | |
|---|---|---|---|---|---|---|---|
| | | Mouse | | Hamster | | Rabbit | |
| Immunogens | Dosage (μg) | A | B | A | B | A | B |
| (1)B2 | 3 × 10 μg | 8 | 16 | 16 | 32 | <8 | 16 |
| (2) B2 | 3 × 30 μg | 8 | 32 | 16 | 32 | <8 | 16 |
| (3) B2 + A1 | 3 × (30 μg + 10 μg) | 512 | 64 | 512 | 128 | 512 | 128 |
| (4) B2 + A1 + alum | 3 × (30 μg + 10 μg) | >1024 | 128 | >1024 | 512 | 1024 | 512 |
| (5) B1 | 3 × 1 μg | 16 | 512 | 32 | 1024 | 32 | 512 |
| (6) B1 | 3 × 3 μg | 16 | >1024 | 32 | >1024 | 64 | >1024 |
| (7) B1 + A1 | 3 × (3 μg + 10 μg) | 512 | >1024 | >1024 | >1024 | 512 | >1024 |
| (8) B1 + A1 | 3 × (10 μg + 10 μg) | 1024 | >1024 | >1024 | >1024 | 1024 | >1024 |
| (9) B1 + A1 | 3 × (30 μg + 10 μg) | >1024 | >1024 | >1024 | >1024 | >1024 | >1024 |
| (10) B1 + A1 + alum | 3 × (10 μg + 10 μg) | >1024 | >1024 | >1024 | >1024 | >1024 | >1024 |
| Toxoid A | 3 × 10 μg | 128 | 16 | 512 | 16 | 1024 | 32 |

* A1 and B1 represent rlipo-A-RBD and rlipo-B-RBD, while B2 represents B-rRBD.
**Antisera were found to be capable of preventing 50% of Vero cell death resulting from either TcdA or TcdB cytotoxicity at the serial dilution.

2.4 A Combination of rlipo-A-RBD (A1) and rlipo-B-RBD (B1) Showed Effective Protection Against C. difficile Spore Challenge in the Hamster Model

To further evaluate the roles of anti-toxin neutralizing antibodies in vivo, hamster challenge model by different strains of C. difficile spore was performed. The hamsters were immunized with different samples, and two weeks after the third immunization, the hamsters were gastrically inoculated with >100 CFU (the dose can kill >50% of challenged hamsters) of C. difficile. After 3 to 4 days, survival rate was determined.

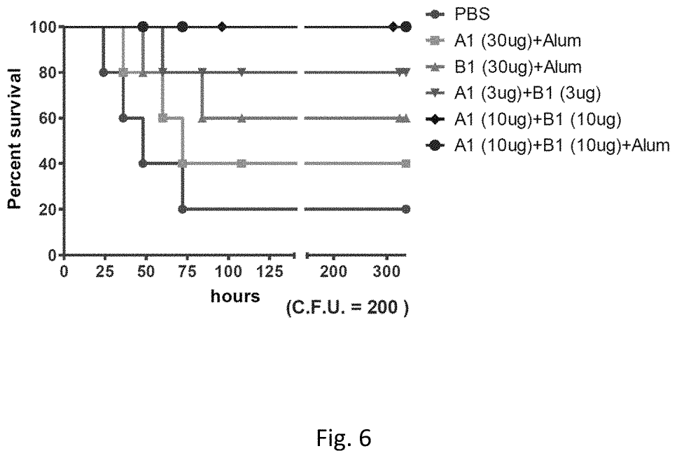

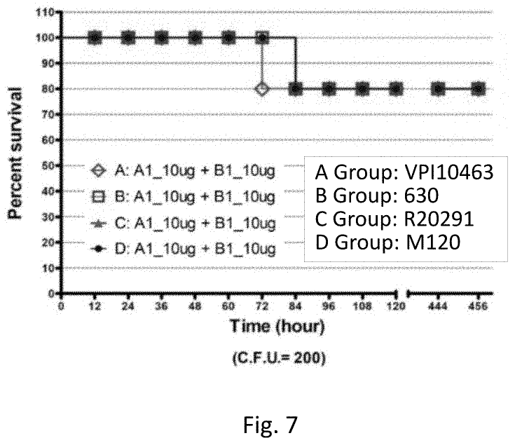

As shown in FIG. 6, most of hamsters died in the groups challenged with VPI10463 strain, and individual rlipo-A-RBD (A1) or individual rlipo-B-RBD (B1), even at a higher dose (30 μg) in the present of alum, could not provide a full protective effect from live spores challenge. In the contrast, a combination of rlipo-A-RBD (A1) and rlipo-B-RBD (B1) induced 80% or higher protective immune responses, even at a relatively lower dose (3 μg), without alum; especially, a combination of rlipo-A-RBD (A1) and rlipo-B-RBD (B1) at the dose of 10 μg for each (A1 10 μg+B1 10 μg), in the absence of alum, exhibited 100% protective effect. Such combination (A1 10 μg+B1 10 μg) was also confirmed effective to provide at least 80% protective effect in other strains such as 630 (a BI/NAP1/027 hyper-virulent strain), M120 and R20291 strains. See FIG. 7.

3. Summary

To sum up, our current vaccine formulation containing a combination of rlipo-A-RBD (A1) and rlipo-B-RBD (B1) elicits strong and consistent neutralizing antibody responses and protection effects against C. difficile spore challenge of various strains in the animal model, and thus should be considered a strong vaccine candidate for CDI vaccine development and future clinical trials.

SEQUENCE INFORMATION

SEQ ID NO: 1 (a nucleic acid sequence encoding a receptor-binding domain of
C. difficile toxin A, VPI10463)
ATGGACAACAAAACCTACTATTACGATGAAGATAGCAAACTGGTAAAAGGCCTGATCAACATCAACAATAGCCTGTTTTAC
TTCGACCCGATCGAGTTTAACCTGGTTACTGGTTGGCAAACCATCAACGGTAAGAAGTATTATTTTGATATCAATACGGGT
GCAGCCCTGACGTCCTACAAAATCATTAACGGCAAACATTTCTATTTCAATAACGACGGTGTTATGCAGCTGGGCGTATTC
AAAGGCCCAGATGGTTTTGAATATTTTGCGCCGGCGAACACCCAGAACAACAACATTGAAGGTCAAGCTATCGTTTACCAG
AGCAAATTCCTGACGCTGAACGGTAAAAAGTACTATTTCGACAACAACTCTAAAGCGGTTACCGGCTGGCGCATCATTAAC
AACGAGAAATACTACTTCAACCCGAACAATGCTATCGCAGCCGTGGGTCTGCAGGTGATTGATAACAACAAGTACTACTTT
AACCCGGACACCGCTATTATTTCTAAAGGTTGGCAGACCGTTAATGGTAGCCGTTATTACTTCGATACCGACACCGCTATC
GCTTTCAACGGTTATAAAACCATCGACGGCAAGCACTTTTATTTCGATTCTGATTGCGTTGTTAAAATCGGCGTGTTCTCC
ACTTCTAACGGTTTTGAATACTTCGCACCGGCAAACACCTACAATAACAATATCGAAGGCCAGGCGATTGTCTACCAGTCC
AAATTTCTGACCCTGAATGGCAAAAAATATTACTTCGACAACAATTCCAAAGCCGTCACCGGTTGGCAGACTATCGACTCT
AAGAAATATTATTTTAACACCAACACTGCGGAAGCAGCAACTGGTTGGCAGACGATTGACGGCAAGAAGTACTATTTTAAC
ACTAACACTGCGGAGGCAGCGACCGGCTGGCAGACCATTGATGGTAAAAAGTATTATTTCAACACTAACACCGCGATTGCA
TCTACCGGTTACACCATCATCAACGGCAAACACTTCTACTTCAACACTGACGGTATCATGCAAATTGGCGTTTTCAAAGGT
CCGAACGGTTTCGAATACTTCGCCCCAGCCAACACGGACGCGAACAACATCGAAGGTCAGGCGATTCTGTACCAGAATGAG
TTCCTGACCCTGAACGGCAAGAAATACTATTTCGGTTCCGATTCCAAAGCTGTAACCGGCTGGCGTATCATCAACAACAAA
AAGTACTACTTCAATCCTAACAACGCAATCGCTGCGATTCACCTGTGTACTATCAACAACGACAAATACTATTTTTCTTAC
GACGGTATCCTGCAGAACGGCTATATCACTATCGAACGTAACAACTTCTATTTCGATGCTAACAACGAAAGCAAAATGGTG
ACGGGCGTGTTCAAAGGCCCGAACGGCTTCGAATAGTTTGCACCTGCAAACACCCACAACAACAACATTGAGGGTCAGGCG
ATCGTTTACCAGAATAAATTCCTGACCCTGAACGGTAAGAAATATTACTTCGATAACGACAGCAAAGCCGTTACCGGCTGG
CAAACCATTGATGGTAAGAAATACTACTTTAATCTGAACACCGCCGAAGCCGCTACTGGTTGGCAGACGATCGATGGCAAA
AAGTACTATTTCAACCTGAATACTGCGGAGGCGGCGACCGGTTGGCAGACCATTGACGGCAAAAAGTATTACTTCAACACG
AACACGTTCATCGCATCCACTGGTTACACCAGCATTAACGGTAAACACTTCTACTTTAACACGGATGGCATTATGCAAATT
GGTGTGTTTAAAGGTCCAAACGGTTTTGAATATTTCGCACCGGCTAACACGGACGCTAACAATATCGAAGGCCAGGCTATT
CTGTACCAAAACAAATTCCTGACTCTGAACGGCAAGAAATATTATTTTGGCTCTGATTCTAAAGCGGTTACGGGCCTGCGT
ACCATCGACGGTAAAAAATACTACTTCAACACCAACACCGCTGTCGCAGTAACTGGCTGGCAGACCATCAACGGTAAAAAG
TACTACTTCAATACCAACACCAGCATCGCTTCTACGGGTTATACTATCATCAGCGGCAAACACTTCTATTTCAACACCGAC
GGTATCATGCAGATCGGCGTTTTCAAAGGTCCGGACGGTTTCGAATACTTCGCTCCGGCGAATACCGACGCCAACAACATC
GAGGGCCAGGCTATCCGTTACCAGAACCGTTTTCTGTATCTGCACGACAATATCTATTACTTCGGTAACAACTCTAAAGCT
GCGACCGGCTGGGTTACGATTGACGGTAACCGCTACTACTTCGAACCGAACACCGCGATGGGTGCGAACGGTTACAAAACC
ATCGATAACAAAAACTTCTATTTCCGTAACGGCCTGCCACAGATCGGTGTTTTCAAGGGTTCTAATGGTTTTGAGTATTTT
GCGCCGGCGAACACTGACGCTAACAACATCGAAGGTCAGGCGATTCGTTATCAGAACCGTTTCCTGCATCTGCTGGGCAAG
ATTTATTACTTCGGCAACAACACCAAAGCGGTGACTGGCTGGCAAACTATTAATGGTAAAGTTTACTACTTTATGCCAGAC
ACTGCTATGGCTGCAGCTGGTGGCCTGTTCGAAATCGACGGCGTTATTTACTTCTTTGGCGTTGACGGCGTGAAAGCGCCG
GGTATTTATGGT
SEQ ID NO: 2 (a receptor-binding domain of C. difficile toxin A peptide, VPI10463)
MDNKTYYYDEDSKLVKGLININNSLFYFDPIEFNLVTGWQTINGKKYYFDINTGAALTSYKIINGKHFYFNNDGVMQLGVF
KGPDGFEYFAPANTQNNNIEGQAIVYQSKFLTLNGKKYYFDNDSKAVTGWRIINNEKYYFNPNNAIAAVGLQVIDNNKYYF
NPDTAIISKGWQTVNGSRYYFDTDTAIAFNGYKTIDGKHFYFDSDCVVKIGVFSTSNGFEYFAPANTYNNNIEGQAIVYQS
KFLTLNGKKYYFDNNSKAVTGWQTIDSKKYYFNTNTAEAATGWQTIDGKKYYFNTNTAEAATGWQTIDGKKYYFNTNTAIA
STGYTIINGKHFYFNTDGIMQIGVFKGPNGFEYFAPANTDANNIEGQAILYQNEFLTLNGKKYYFGSDSKAVTGWRIINNK
KYYFNPNNAIAAIHLCTINNDKYYFSYDGILQNGYITIERNNFYFDANNESKMVTGVFKGPNGFEYFAPANTHNNNIEGQA
IVYQNKFLTLNGKKYYFDNDSKAVTGWQTIDGKKYYFNLNTAEAATGWQTIDGKKYYFNLNTAEAATGWQTIDGKKYYFNT
NTFIASTGYTSINGKHFYFNTDGIMQIGVFKGPNGFEYFAPANTDANNIEGQAILYQNKFLTLNGKKYYFGSDSKAVTGLR
TIDGKKYYFNTNTAVAVTGWQTINGKKYYFNTNTSIASTGYTIISGKHFYFNTDGIMQIGVFKGPDGFEYFAPANTDANNI
EGQAIRYQNRFLYLHDNIYYFGNNSKAATGWVTIDGNRYYFEPNTAMGANGYKTIDNKNFYFRNGLPQIGVFKGSNGFEYF
APANTDANNIEGQAIRYQNRFLHLLGKIYYFGNNSKAVTGWQTINGKVYYFMPDTAMAAAGGLFEIDGVIYFFGVDGVKAP
GIYG
SEQ ID NO: 3 (a nucleic acid sequence encoding a receptor-binding domain of
C. difficile toxin B peptide, VPI10463)
ATGATGGTTTCTGGCCTGATCTATATTAACGATTCTCTGTACTATTTCAAACCGCCGGTTAACAACCTGATCACCGGTTTC
GTAACTGTTGGCGACGATAAGTACTACTTTAATCCAATTAACGGTGGTGCGGCCAGCATTGGCGAAACTATCATCGACGAC
AAAAACTACTACTTCAATCAGTCCGGTGTTCTGCAGACCGGTGTATTCTCTACCGAAGATGGCTTCAAGTATTTTGCGCCG
GCTAACACCCTGGATGAAAACCTGGAAGGTGAAGCTATTGACTTTACCGGCAAACTGATCATTGACGAAAATATCTACTAC
TTCGACGATAACTACCGTGGTGCAGTAGAATGGAAAGAACTGGACGGTGAAATGCACTACTTCTCTCCGGAAACTGGTAAA
GCTTTTAAGGGCCTGAACCAGATCGGTGATTACAAATACTACTTCAACAGCGATGGTGTGATGCAAAAAGGTTTCGTGAGC
ATCAATGATAACAAGCACTACTTTGACGATTCCGGCGTGATGAAAGTCGGCTATACTGAAATCGACGGTAAACACTTCTAC
TTTGCTGAAAACGGCGAAATGCAAATCGGCGTTTTCAATACTGAAGACGGCTTCAAATATTTCGCTCATCACAACGAAGAT
CTGGGTAACGAAGAAGGCGAGGAGATTTCCTACTCCGGCATTCTGAACTTCAACAACAAAATTTACTACTTTGATGATTCT
TTCACCGCCGTGGTTGGCTGGAAAGACCTGGAAGATGGTTCCAAATATTATTTCGATGAGGATACCGCTGAAGCGTACATT
GGTCTGTCTCTGATCAACGATGGCCAGTACTATTTTAACGACGACGGCATTATGCAGGTGGGCTTCGTTACCATTAACGAC
AAAGTTTTCTATTTTAGCGATTCCGGTATCATCGAAAGCGGCGTGCAGAACATCGATGATAACTACTTCTACATCGATGAC
AATGGTATCGTTCAGATCGGCGTATTCGATACCTCTGACGGTTATAAATACTTCGCCCCGGCGAACACCGTTAACGACAAC
ATTTATGGTCAGGCAGTCGAGTACTCCGGCCTGGTTCGTGTGGGTGAAGACGTATACTACTTCGGTGAAACGTACACCATC
GAGACGGGCTGGATCTACGACATGGAAAACGAATCTGACAAATATTACTTCAACCCGGAAACTAAAAAGGCGTGCAAAGGC
ATCAACCTGATCGATGACATCAAATACTATTTCGACGAAAAAGGCATCATGCGCACTGGTCTGATCAGCTTCGAAAACAAC
AACTATTATTTCAACGAGAATGGCGAGATGCAGTTCGGTTACATCAACATCGAAGACAAGATGTTCTACTTTGGTGAAGAC
GGTGTAATGCAGATTGGTGTTTTCAACACCCCTGACGGTTTTAAATATTTTGCACATCAGAACACTCTGGACGAGAACTTC
GAGGGTGAGTCTATCAACTATACCGGCTGGCTGGACCTGGATGAGAAACGTTACTACTTCACGGACGAATACATTGCGGCA
ACCGGTAGCGTGATCATTGATGGTGAAGAATACTATTTTGATCCGGACACGGCACAACTGGTCATCTCTGAA
SEQ ID NO: 4 (a receptor-binding domain of C. difficile toxin B peptide, VPI10463)
MMVSGLIYINDSLYYFKPPVNNLITGFVTVGDDKYYFNPINGGAASIGETIIDDKNYYFNQSGVLQTGVFSTEDGFKYFAP
ANTLDENLEGEAIDFTGKLIIDENIYYFDDNYRGAVEWKELDGEMHYFSPETGKAFKGLNQIGDYKYYFNSDGVMQKGFVS
INDNKHYFDDSGVMKVGYTEIDGKHFYFAENGEMQIGVFNTEDGFKYFAHHNEDLGNEEGEEISYSGILNFNNKIYYFDDS
FTAVVGWKDLEDGSKYYFDEDTAEAYIGLSLINDGQYYFNDDGIMQVGFVTINDKVFYFSDSGIIESGVQNIDDNYFYIDD
NGIVQIGVFDTSDGYKYFAPANTVNDNIYGQAVEYSGLVRVGEDVYYFGETYTIETGWIYDMENESDKYYFNPETKKACKG
INLIDDIKYYFDEKGIMRTGLISFENNNYYFNENGEMQFGYINIEDKMFYFGEDGVMQIGVFNTPDGFKYFAHQNTLDENF
EGESINYTGWLDLDEKRYYFTDEYIAATGSVIIDGEEYYFDPDTAQLVISE
AMINO ACIDS SEQUENCE OF LIPIDATED TOXIN A-RBD (rLIPO-A-RBD)
SEQ ID NO: 5
CSQEAKQEVKEAVQAVESDVKDTAGSHMDNKTYYYDEDSKLVKGLININNSLFYFDPIEFNLVTGWQTINGKKYYFDINTG
AALTSYKIINGKHFYFNNDGVMQLGVFKGPDGFEYFAPANTQNNNIEGQAIVYQSKFLTLNGKKYYFDNNSKAVTGWRIIN
NEKYYFNPNNAIAAVGLQVIDNNKYYFNPDTAIISKGWQTVNGSRYYFDTDTAIAFNGYKTIDGKHFYFDSDCVVKIGVFS
TSNGFEYFAPANTYNNNIEGQAIVYQSKFLTLNGKKYYFDNNSKAVTGWQTIDSKKYYFNTNTAEAATGWQTIDGKKYYFN
TNTAEAATGWQTIDGKKYYFNTNTAIASTGYTIINGKHFYFNTDGIMQIGVFKGPNGFEYFAPANTDANNIEGQAILYQNE
FLTLNGKKYYFGSDSKAVTGWRIINNKKYYFNPNNAIAAIHLCTINNDKYYFSYDGILQNGYITIERNNFYFDANNESKMV
TGVFKGPNGFEYFAPANTHNNNIEGQAIVYQNKFLTLNGKKYYFDNDSKAVTGWQTIDGKKYYFNLNTAEAATGWQTIDGK
KYYFNLNTAEAATGWQTIDGKKYYFNTNTFIASTGYTSINGKHFYFNTDGIMQIGVFKGPNGFEYFAPANTDANNIEGQAI
LYQNKFLTLNGKKYYFGSDSKAVTGLRTIDGKKYYFNTNTAVAVTGWQTINGKKYYFNTNTSIASTGYTIISGKHFYFNTD
GIMQIGVFKGPDGFEYFAPANTDANNIEGQAIRYQNRFLYLHDNIYYFGNNSKAATGWVTIDGNRYYFEPNTAMGANGYKT
IDNKNFYFRNGLPQIGVFKGSNGFEYFAPANTDANNIEGQAIRYQNRFLHLLGKIYYFGNNSKAVTGWQTINGKVYYFMPD
TAMAAAGGLFEIDGVIYFFGVDGVKAPGIYGLE
(the underlined refers to a lipid-box signal sequence, SEQ ID NO: 7)
AMINO ACIDS SEQUENCE OF rLIPO-B-RBD SEQ ID NO: 6
CSQEAKQEVKEAVQAVESDVKDTAGSHMMVSGLIYINDSLYYFKP PVNNLITGFVTVGDDKYYFNPINGGAASIGETIID
DKNYYFNQSGVLQTGVFSTEDGFKYFAPANTLDENLEGEAIDFTGKLIIDENIYYFDDNYRGAVEWKELDGEMHYFSPETG
KAFKGLNQIGDYKYYFNSDGVMQKGFVSINDNKHYFDDSGVMKVGYTEIDGKHFYFAENGEMQIGVFNTEDGFKYFAHHNE
DLGNEESEEISYSGILNFNNKIYYFDDSFTAVVGWKDLEDGSKYYFDEDTAEAYIGLSLINDGQYYFNDDGIMQVGFVTIN
DKVFYFSDSGIIESGVQNIDDNYFYIDDNGIVQIGVFDTSDGYKYFAPANTVNDNIYGQAVEYSGLVRVGEDVYYFGETYT
IETGWIYDMENESDKYYFNPETKKACKGINLIDDIKYYFDEKGIMRTGLISFENNNYYFNENGEMQFGYINIEDKMFYFGE
DGVMQIGVFNTPDGFKYFAHQNTLDENFEGESINYTGWLDLDEKRYYFTDEYIAATGSVIIDGEEYYFDPDTAQLVISELE
(the underlined refers to a lipid-box signal sequence, SEQ ID NO: 7)

### REFERENCE

1. Knoop F C, Owens M, Crocker I C. Clostridium difficile: clinical disease and diagnosis. Clin Microbiol Rev. 1993; 6(3):251-65.
2. Lyerly D M, Krivan H C, Wilkins T D. Clostridium difficile: its disease and toxins. Clin Microbiol Rev. 1988; 1(1):1-18.
3. Kelly C P, LaMont J T. Clostridium difficile—more difficult than ever. N Engl J Med. 2008; 359(18):1932-40.
4. McDonald L C, Killgore G E, Thompson A, Owens R C, Kazakova S V, Sambol S P, et al. An epidemic, toxin gene-variant strain of Clostridium difficile. N Engl J Med. 2005; 353(23):2433-41.
5. He M, Miyajima F, Roberts P, Ellison L, Pickard D J, Martin M J, et al. Emergence and global spread of epidemic healthcare-associated Clostridium difficile. Nat Genet. 2013; 45(1):109-13.
6. Sun X, Savidge T, Feng H. The enterotoxicity of Clostridium difficile toxins. Toxins. 2010; 2(7):1848-80.
7. Kuehne S A, Cartman S T, Heap J T, Kelly M L, Cockayne A, Minton N P. The role of toxin A and toxin B in Clostridium difficile infection. Nature. 2010; 467(7316):711-3.
8. Davies A H, Roberts A K, Shone C C, Acharya K R. Super toxins from a super bug: structure and function of Clostridium difficile toxins. Biochem J. 2011; 436(3):517-26.
9. Fernie D S, Thomson R O, Batty I, Walker P D. Active and passive immunization to protect against antibiotic associated caecitis in hamsters. Dev Biol Stand. 1983; 53:325-32.
10. Kim P H, Iaconis J P, Rolfe R D. Immunization of adult hamsters against Clostridium difficile-associated ileocecitis and transfer of protection to infant hamsters. Infect Immun. 1987; 55(12):2984-92.
11. Kyne L, Warny M, Qamar A, Kelly C P. Association between antibody response to toxin A and protection against recurrent Clostridium difficile diarrhoea. Lancet. 2001; 357(9251):189-93.
12. Leav B A, Blair B, Leney M, Knauber M, Reilly C, Lowy I, et al. Serum anti-toxin B antibody correlates with protection from recurrent Clostridium difficile infection. Vaccine. 2010; 28(4):965-9.
13. Davies N L, Compson J E, Mackenzie B, O'Dowd V L, Oxbrow A K, Heads J T, et al. A mixture of functionally oligoclonal humanized monoclonal antibodies that neutralize Clostridium difficile TcdA and TcdB with high levels of in vitro potency shows in vivo protection in a hamster infection model. Clin Vaccine Immunol. 2013; 20(3):377-90.
14. Marozsan A J, Ma D, Nagashima K A, Kennedy B J, Kang Y K, Arrigale R R, et al. Protection against Clostridium difficile infection with broadly neutralizing antitoxin monoclonal antibodies. J Infect Dis. 2012; 206(5):706-13.
15. Babcock G J, Broering T J, Hernandez H J, Mandell R B, Donahue K, Boatright N, et al. Human monoclonal antibodies directed against toxins A and B prevent Clostridium difficile-induced mortality in hamster. Infect Immun. 2006; 74(11):6339-47.
16. Kink J A, Williams J A. Antibodies to recombinant Clostridium difficile toxins A and B are an effective treatment and prevent relapse of C. difficile-associated disease in a hamster model of infection. Infect Immun. 1998; 66(5):2018-25.
17. Wang H, Sun X, Zhang Y, Li S, Chen K, Shi L, et al. A chimeric toxin vaccine protects against primary and recurrent Clostridium difficile infection. Infect Immun. 2012; 80(8):2678-88.
18. Anosova N G, Brown A M, Li L, Liu N, Cole L E, Zhang J, et al. Systemic antibody responses induced by a two-component Clostridium difficile toxoid vaccine protect against C. difficile-associated disease in hamsters. J med microbiol. 2013; 62(Pt9):1394-404.
19. Torres J F, Lyerly D M, Hill J E, Monath T P. Evaluation of formalin-inactivated Clostridium difficile vaccines administered by parenteral and mucosal routes of immunization in hamsters. Infect Immun. 1995; 63(12):4619-27.
20. Sauerborn M, Leukel P, von Eichel-Streiber C. The C-terminal ligand-binding domain of Clostridium difficile toxin A (TcdA) abrogates TcdA-specific binding to cells and prevents mouse lethality. FEMS Microbiol Lett. 1997; 155(1):45-54.
21. Seregin S S, Aldhamen Y A, Rastall D P, Godbehere S, Amalfitano A. Adenovirus-based vaccination against Clostridium difficile toxin A allows for rapid humoral immunity and complete protection from toxin A lethal challenge in mice. Vaccine. 2012; 30(8):1492-501.
22. Tian J H, Fuhrmann S R, Kluepfel-Stahl S, Carman R J, Ellingsworth L, Flyer D C. A novel fusion protein containing the receptor binding domains of C. difficile toxin A and toxin B elicits protective immunity against lethal toxin and spore challenge in preclinical efficacy models. Vaccine. 2012; 30(28):4249-58.
23. Ryan E T, Butterton J R, Smith R N, Carroll P A, Crean T I, Calderwood S B. Protective immunity against Clostridium difficile toxin A induced by oral immunization with a live, attenuated Vibrio cholerae vector strain. Infect Immun. 1997; 65(7):2941-9.
24. Ward S J, Douce G, Figueiredo D, Dougan G, Wren B W. Immunogenicity of a Salmonella typhimurium aroA aroD vaccine expressing a nontoxic domain of Clostridium difficile toxin A. Infect Immun. 1999; 67(5):2145-52.
25. Gardiner D F, Rosenberg T, Zaharatos J, Franco D, Ho D D. A DNA vaccine targeting the receptor-binding domain of Clostridium difficile toxin A. Vaccine. 2009; 27(27):3598-604.
26. Permpoonpattana P, Hong H A, Phetcharaburanin J, Huang J M, Cook J, Fairweather N F, et al. Immunization with Bacillus spores expressing toxin A peptide repeats protects against infection with Clostridium difficile strains producing toxins A and B. Infect Immun. 2011; 79(6):2295-302.
27. Ho J G, Greco A, Rupnik M, Ng K K. Crystal structure of receptor-binding C-terminal repeats from Clostridium difficile toxin A. Proc Natl Acad Sci USA. 2005; 102(51):18373-8.
28. Dove C H, Wang S Z, Price S B, Phelps C J, Lyerly D M, Wilkins T D, et al. Molecular characterization of the Clostridium difficile toxin A gene. Infect Immun. 1990; 58(2):480-8.
29. Krivan H C, Clark G F, Smith D F, Wilkins T D. Cell surface binding site for Clostridium difficile enterotoxin: evidence for a glycoconjugate containing the sequence Gal alpha 1-3Gal beta 1-4GlcNAc. Infect Immun. 1986; 53(3):573-81.
30. Huang J H, Shen Z Q, Lien S P, Hsiao K N, Leng C H, Chen C C, et al. Biochemical characterizations of the receptor binding domains of C. difficile Toxin A. PLoS ONE 2015 (revision manuscript PONE-D-14-43934R1).
31. Huang J H, Wu C W, Lien S P, Hsiao K N, Leng C H, Lin Y H, et al. Biochemical and immunological characterizations of the receptor binding domain of C. difficile toxin B. J Vaccines Vaccin. 2015; 6(2): 276.
32. Chen H W, Liu S J, Liu H H, Kwok Y, Lin C L, Lin L H, et al. A novel technology for the production of a heterologous lipoprotein immunogen in high yield has implications for the field of vaccine design. Vaccine. 2009; 27(9):1400-9.
33. Lutz M B, Kukutsch N, Ogilvie A L, Rossner S, Koch F, Romani N, et al. An advanced culture method for generating large quantities of highly pure dendritic cells from mouse bone marrow. J Immunol Methods. 1999; 223(1):77-92.
34. Lyras D, O'Connor J R, Howarth P M, Sambol S P, Carter G P, Phumoonna T, et al. Toxin B is essential for virulence of Clostridium difficile. Nature. 2009; 458(7242):1176-9.
35. Hussack G, Arbabi-Ghahroudi M, van Faassen H, Songer J G, Ng K K, Mackenzie R, et al. Neutralization of Clostridium difficile toxin A with single-domain antibodies targeting the cell receptor binding domain. J Biol Chem. 2011; 286(11):8961-76.

What is claimed is:

1. An immunogenic preparation against Clostridium difficile infection (CDI), comprising (i) a lipidated receptor-binding domain of C. difficile toxin A (lipo-A-RBD) polypeptide and (ii) a lipidated receptor-binding domain of C. difficile toxin B (lipo-B-RBD) polypeptide, in an amount effective to induce protective immunity against CDI.

2. The immunogenic preparation of claim 1, wherein the lipo-A-RBD polypeptide comprises a receptor-binding domain of C. difficile toxin A (A-RBD) polypeptide modified with a first lipid moiety, and/or the lipo-B-RBD polypeptide comprises a receptor-binding domain of C. difficile toxin B (B-RBD) polypeptide modified with a second lipid moiety.

3. The immunogenic preparation of claim 2, wherein the first lipid moiety and the second lipid moiety are different or the same.

4. The immunogenic preparation of claim 2, wherein each of the lipid moieties comprises one or more lipid molecules selected from the group consisting of palmitoyl, stearoyl, decanoyl, and any combination thereof.

5. The immunogenic preparation of any of claim 1, wherein the A-RBD polypeptide comprises an amino acid sequence at least 85% identical to SEQ ID No: 2, preferably SEQ ID NO: 2.

6. The immunogenic preparation of any of claim 1, wherein the B-RBD polypeptide comprises an amino acid sequence at least 85% identical to SEQ ID No: 4, preferably SEQ ID NO: 4.

7. The immunogenic preparation of any of claim 1, which further comprises a pharmaceutically acceptable carrier.

8. The immunogenic preparation of any of claim 1, which further comprise an adjuvant.

9. The immunogenic preparation of any of claim 1, which does not include a further component as an adjuvant.

10. A method for generating protective immunity against CDI in a subject in need, comprising administering to the subject an effective amount of a preparation comprising (i) a lipidated receptor-binding domain of C. difficile toxin A (lipo-A-RBD) polypeptide and (ii) a lipidated receptor-binding domain of C. difficile toxin B (lipo-B-RBD) polypeptide.

11. The method of claim 10, which is effective in treating or preventing a disease or disorder associated with CDI.

12. The method of claim 11, wherein the disease or disorder is selected from the group consisting of diarrhea, pseudomembranous colitis, and toxic megacolon.
